(19) United States
(12) Patent Application Publication
(10) Pub. No.: US 2017/0238788 A1
(43) Pub. Date: Aug. 24, 2017

(54) Illuminated Endoscopic Pedicle Probe With Dynamic Real Time Monitoring For Proximity To Nerves

(71) Applicant: Avery M. Jackson, III, Grand Blanc, MI (US)

(72) Inventor: Avery M. Jackson, III, Grand Blanc, MI (US)

(21) Appl. No.: 15/269,327

(22) Filed: Sep. 19, 2016

Related U.S. Application Data
(60) Provisional application No. 62/219,798, filed on Sep. 17, 2015.

(51) Int. Cl. A61B 1/00; A61B 1/015; A61B 5/0488; A61B 5/11; A61B 1/05; A61B 1/06

(52) U.S. Cl. CPC A61B 1/00087; A61B 1/05; A61B 1/06; A61B 5/0488; A61B 5/1107; A61B 1/015; A61B 1/00045

(57) ABSTRACT

An endoscopic pedicle probe has an elongate body with a proximal end and a distal end. A tip on the distal end is pushed into the pedicle to form the hole, and an enlarged head on the proximal end enables a surgeon to manipulate the probe. The body has an inner shaft with a cylindrical sleeve telescopically engaged over it. An endoscope extends through a longitudinal bore in the shaft, with a camera at the tip end connected with a monitor to enable a surgeon to visually observe the area being treated. A light extends through another bore to illuminate the area, and a further bore conducts irrigation fluid to and from the area. The sleeve is made of electrically non-conductive material and the shaft and tip are made of electrically conductive material to enable stimulation of nerves at the treatment area.

This application claims the benefit of provisional application Ser. No. 62/219,798, filed Sep. 17, 2015, and is a continuation-in-part of U.S. patent application Ser. No. 14/723,067, filed May 27, 2015, which is a continuation-in-part of application Ser. No. 14/289,795, filed May 29, 2014, which claims the benefit of U.S. provisional patent application Ser. No. 61/955,895, filed Mar. 20, 2014, and is a continuation-in-part of U.S. patent application Ser. No. 13/728,987, filed Dec. 27, 2012, which in turn claims the benefit of U.S. provisional patent application Ser. No. 61/647,747, filed May 16, 2012.

### TECHNICAL FIELD

This invention relates generally to surgical instruments. More specifically, the invention relates to a pedicle probe for use in forming holes in a vertebral pedicle in preparation for pedicle screw insertion. According to one feature of the invention the probe incorporates at least one endoscope to enable the surgeon to see the area being treated. A light is integrated with the probe to illuminate the area being treated, and in a preferred embodiment irrigation means is associated with the probe to flush debris away from the area being treated to prevent the view from being obstructed. In accordance with a further preferred embodiment the probe is provided with mechanomyography (MMG) or electromyography (EMG) capability to alert the surgeon if the pedicle is about to be breached. In another embodiment a replaceable tip is provided on the distal end of the probe, and in a still further preferred embodiment the entire probe is disposable. The probe of the invention may have any one or any combination of these features.

### BACKGROUND ART

It is sometimes necessary to perform surgery on the spine in order to repair trauma, correct a deformity, or alleviate the effects of disease. Spinal fusion or stabilization is one procedure that may be employed to treat these conditions. According to one source, at the present time there are approximately 30 million spine procedures performed globally each year, including approximately 400,000 cervical and lumbar fixations performed in the US.

Spinal fusion may be accomplished by insertion of screws into the pedicle to stabilize a spinal segment. The pedicle is a dense, stem-like structure projecting from the posterior of a vertebra, and there are two pedicles per vertebra that connect to other structures. Since the pedicle is the strongest point of attachment of the spine, significant forces can be applied to the spine without failure of the bone-to-metal connection.

To insert pedicle screws, a long, thin, metal probe is inserted through the pedicle and into the vertebral body, forming a hole for reception of the screw. Conventional pedicle probes may be straight or curved, and comprise an elongate solid metal shaft with an enlarged hand grip on the proximal end. The probe may have a shaped distal end adapted for forming a hole through the pedicle, or a separate awl or reamer may first be used to form a hole through the pedicle, and the probe then inserted into the cancellous bone of the pedicle and into the vertebral body to develop a path for the screw. A variety of probes are known in the prior art, including the so-called gear shift pedicle probe and the Fox pedicle probe. The gear shift probe has a round head on its proximal end, whereas the Fox probe has a flat disc-shaped head on its proximal end.

Most conventional modalities used to approximate or simulate screw placement are indirect, and include fluoroscopic guidance and frameless stereotactic guidance. Approximations of the pedicle and surrounding vital structures are obtained from a CT scan or MRI done prior to surgery.

Proper positioning of a conventional probe depends to an extent upon tactile feel.

For instance, advancement of the probe should be smooth and consistent. A sudden plunge suggests breaking out of the pedicle laterally, and an increase in resistance indicates abutment against the pedicle or vertebral body cortex.

These conventional modalities require a steep learning curve, and improper or inaccurate manipulation of the probe and placement of the pedicle screw can result in caudal or medial penetration of the pedicle cortex and dural or neural injury.

With conventional pedicle probes there is no direct way to confirm that the hole was made within the pedicle and that the screw will be placed completely inside the pedicle. Surrounding structures can be injured if a portion of the screw is placed outside of the pedicle. There can be nerve root injury, epidural vessel injury, or spinal fluid leakage caused by a misplaced screw.

The rate of misplaced pedicle screws as reported in the literature ranges from a few percent to forty percent, and the rate of permanent neurological deficits as reported in the literature ranges from 2% to 5%. These deficits can result in post-surgery pain, lifetime injury, and loss of confidence by the surgeon. Accountable care also can lead to lawsuits.

Many conventional hand-held devices are simple and have relatively low cost. They can be optics and/or ultrasound based and some have haptic or auditory feedback. However, background noise in an operating room environment impedes the effectiveness of devices having auditory feedback, and conventional devices typically have inferior ergonomics. There can be proprioception confusion of turgor, and there is absence of debris management and neuro-monitoring.

Other conventional devices have navigation systems that provide an indication of approximate anatomy based on integrated haptics, imaging modalities and neuromonitoring. However, these conventional devices are expensive and do not provide real-time monitoring.

Applicant's earlier U.S. Pat. No. 6,855,105, discloses an endoscopic pedicle probe having a camera at its distal end connected with an endoscopic monitor via a fiber optic bundle extending through the probe to provide the surgeon with a view of the area being treated, thus overcoming many of the shortcomings of conventional pedicle probes.

Recognizing that illumination of the area being treated would greatly enhance the usefulness of the endoscope, in his earlier U.S. patent application, Ser. No. 13/728,987, applicant added a light to illuminate the area being treated. Applicant also added irrigation means to flush debris away from the area to so that the view of the endoscope camera is not obstructed.

In applicant's prior provisional patent application Ser. No. 61/955,895, an endoscope and light are combined in a single unit that can be extended through a single bore in the probe, thus reducing the number of bores required and simplifying the construction of the probe.

Although the earlier embodiments of applicant's invention cured many of the deficiencies of prior art probes, it was difficult for the surgeon to know when a breach was about to occur due to misplacement of the probe.

U.S. Pat. No. 8,255,044 discloses a system that uses the principles of electromyography to alert the surgeon when a breach is about to occur and potentially cause damage to nerves. The system in that patent takes advantage of the insulating characteristics of the walls of the pedicle and the conductivity of adjacent nerve roots and uses electromyographic monitoring to perform dynamic pedicle integrity assessments to detect a breach or potential breach of the pedicle and alert the surgeon. The system in the '044 patent involves establishing electrical communication between a stimulation source and the interior of a pedicle hole during the hole formation, hole preparation, and/or screw introduction steps of pedicle screw fixation. By applying a stimulation signal during these steps and monitoring the neuromuscular responses resulting from this stimulation, the system automatically detects and communicates to the user whether the integrity of the pedicle has been compromised, i.e. breached or about to be breached. The probe in this patent is made of electrically conductive material and is connected with a source of electrical energy to apply an electric field to the probe. A plunger 41 is manually applied to a device 65 to establish electrical connection with the source of electrical energy. To avoid shunting between the conductive walls of the probe and adjacent tissue when the stimulation signal is applied, a flexible insulating sheath is placed around the probe body.

Recent advances in lateral access spinal fusion surgery techniques now enable surgeons to perform minimally invasive lateral access spinal fusion in a safe and effective muscle-sparing manner Traditional posterior fusion techniques require the dissection and retraction of back muscles, bones, vessels, ligaments, and nerves; whereas traditional anterior approaches through the abdominal musculature risk injury to major vascular structures such as the aorta and iliac vessels, as well as the very delicate genitourinary structures.

In the new lateral transpsoas approach access is through the side of the patient and through the psoas muscle, using mechanomyography (MMG) to provide dynamic real-time monitoring of the location of nerves. MMG functions by measuring the mechanical response of muscle following nerve stimulation, compared to traditional electromyography (EMG) techniques that monitor the electrical response of muscle and are therefore subject to the potential for electrical interference. MMG has a faster response than EMG, indicating a higher sensitivity for detection of nerves at a lower threshold. Muscle response to electrical stimulus varies with the distance of the nerve from the source of the stimulus and MMG can tell the surgeon exactly how far away he or she is from the nerve. Working with different levels of current, the surgeon is able to establish a relationship between the current and distance, allowing the surgeon to determine precisely how far a nerve is from the stimulus probe.

MMG detects the presence of a nerve on average 1.2 seconds earlier than EMG, using approximately half the amount of stimulating current. Since electrical resistance is highly variable, depending on the conducting tissue, EMG monitoring systems may utilize currents as high as 200 mA. The MMG system typically has a maximum current output of 6 mA, nearly 35 times less than comparable EMG systems.

MMG is a more sensitive indicator for locating nerves, and the surgeon can, without looking, know within a millimeter or two where he or she is in relation to the nerve. By utilizing a system that requires less electrical current, the surgeon is able to further decrease the risk of injury to patients.

Sentio, LLC of Wixom Mich. has developed a mechanomyography (MMG) surgical access tool for locating and mapping motor nerve roots and their peripheral extensions during lateral access spinal fusion surgery. The Sentio MMG system adheres accelerometer sensors to the surface of the skin directly over muscles innervated by the nerves the surgeon wishes to identify. A stimulator probe is manipulated by the surgeon about the surgical site to stimulate for the presence of motor nerves. When a nerve is identified, the surgeon is provided with a “stop” alert. At any time the surgeon is stimulating and receiving a “go” alert, the surgeon can infer that a “go” alert when using stimulation current at:
1 mA means the Sentio probe is at least 1 mm from the nerve;
5 mA means the Sentio probe is at least 5 mm from the nerve;
15 mA means the Sentio probe is at least 15 mm from the nerve.

Sentio MMG® measures the same physiological phenomena associated with muscle contraction as EMG, but does so via mechanical means as opposed to electrical. MMG does not involve needles, therefore reducing the risk of needle sticks to the surgeon and operating room (OR) staff and further reducing the chance of infection to the patient and OR personnel; does not require any skin prep; and readings require only a single sensor patch to be adhered to the skin, whereas EMG requires three electrode areas to be prepared.

In use of the Sentio system, an incision is made in the side of the patient and the surgeon inserts a dilator through the incision and to the level of the spine. A small electrical signal is sent through the dilator to stimulate nerves and guide the surgeon in placement of the dilator directly over the disc space and in front of the lumbar nerve structures. This system does not involve the use of a pedicle probe or placement of pedicle screws.

It would be advantageous to have a pedicle probe that could use mechanomyography or electromyography to stimulate and monitor neuromuscular responses during a procedure for pedicle screw placement without having to incorporate the flexible insulating shield and plunger used in the U.S. Pat. No. 8,255,044 patent.

### SUMMARY OF THE INVENTION

The present invention is an intuitive and ergonomic pedicle probe incorporating state of the art scope technology and robust functionality. It uses mechanomyography or electromyography to monitor neuromuscular responses during a procedure for pedicle screw placement and provides real-time visual confirmation of screw trajectory and pedicle soundness. Use of the probe of the invention results in decreased radiation exposure compared to other pedicle screw placement technologies, and provides enhanced quality of care with the ability to document pedicle integrity.

The probe of the invention has integrated visualization, illumination, irrigation, aspiration, and neuromonitoring. This allows the surgeon to detect a potential breach before it occurs, and the extent and location of any breach that does occur during pedicle cannulation. The probe is compatible with common arthroscopy operating room equipment. In a preferred embodiment it is a disposable single-use pedicle probe.

For the surgeon, the probe of the invention enables real-time visualization of the surgical site during surgery and increases accuracy of pedicle screw placement. It decreases radiation exposure due to use of a C-arm, is easy to use, and minimizes litigation risk.

For the patient, the probe of the invention significantly lowers the chance of injury to the spinal cord and nerves, minimizes radiation exposure, enables less time under anesthesia, and provides a better surgical outcome.

In accordance with a preferred embodiment of pedicle probe according to the invention, the probe comprises an elongate shaft of conductive material shielded by a sleeve or sheath of non-conductive material telescopically engaged on the shaft. A conductive tip is attached to the distal end of the shaft so that neuromuscular response can be induced at the target site by supplying electrical energy to the shaft and tip.

In use, particularly of an MMG system such as that by Sentio LLC, a connector such as an alligator clip can be attached to the probe so that the Sentio system can supply electrical energy to the probe. The invention provides the surgeon with a warning when a nerve is approached or a breach is about to occur so that the surgeon can adjust the position of the probe and avoid a breach and/or contact with a nerve.

The tip is securely removably attached to the distal end of the shaft of the probe and can be replaced when worn or damaged or when a tip having different characteristics is desired. Applicant's earlier application Ser. No. 13/728,987 added a replaceable tip enabling a new or different tip to be used without having to replace the entire instrument.

Rather than separate components, a light and endoscope can be incorporated together in a single unit, thus requiring only a single bore extending longitudinally through the probe to accommodate these two features. The endoscope and light provide the surgeon with a visual indication of the position of the probe relative to the pedicle and surrounding structure during a surgical procedure, enabling the surgeon to directly confirm the location of the probe and ensuring accurate placement of the hole for receiving a pedicle screw.

Irrigation means associated with the probe flushes the area being treated with a fluid, such as, e.g. saline, to remove body fluids and debris that might otherwise obscure the view.

One suitable endoscope incorporating a light is the Medigus LEDprobe, an integrated camera and illumination device available from Medigus, Ltd. of Omer, Israel. The Medigus LEDprobe is a 1.8/2.0 mm diameter rigid endoscope which includes a 1.2 mm camera in the distal tip of the device. It is equipped with high quality 100°/140° field of view (FOV) optics and a large LED located in the handle of the device. The device has a stainless steel shaft and illumination is led through the shaft towards the distal tip of the device where the camera is located via fiber-for-illumination. The LED is powered by the video processor and, therefore, no additional peripherals are required other than a monitor. The camera used with this system has a diameter of only 1.2 mm and a length of only 5 mm. It has high quality 100 degree FOV optics and a shielded camera cable with a metal connector as well as a video processor.

The endoscopic pedicle probe of the invention puts the surgeon “in the pedicle” with the use of endoscopy and avoids breaches by using EMG or MMG. The positioning of the probe can be directly and accurately visually observed during surgery, whereby the surgeon can avoid placing the screw too medial, lateral, cranial, caudal, or deep. The surgeon will know if the wall of the pedicle is about to be breached, and the position of the probe can be adjusted to avoid a breach. The surgeon can also avoid parallax that may cause errors when using fluoroscopic guidance.

The probe of the invention will not represent an additional instrument needed for pedicle screw placement. Accordingly, there will be no additional costs or equipment needed to perform the standard spinal fusion.

The probe of the invention can be utilized in the cervical spine for lateral mass screw placement, pedicle screw placement, or trans-articular screw placement. It can be used in the thoracic, lumbar, and sacral spine for pedicle screw placement and trans-laminar screw placement, and can be used in standard open spine fusion or in minimally invasive percutaneous spine fusion.

### BRIEF DESCRIPTION OF THE DRAWINGS

The foregoing as well as other objects and advantages of the invention will become apparent from the following detailed description when considered in conjunction with the accompanying drawings, wherein like reference characters designate like parts throughout the several views, and wherein:

FIG. 1 is an isometric view of a typical prior art device.

FIG. 2 depicts a system incorporating a probe with MMG or EMG capability according to the invention.

FIG. 3 is an enlarged fragmentary view of the pedicle probe of the invention, looking at an angle from slightly above and positioned to form a hole in a pedicle.

FIG. 4 is an enlarged fragmentary view of the proximal end of the probe of the invention, looking at a slight angle from above.

FIG. 5A is an enlarged fragmentary isometric view showing one side of the distal end of the probe of the invention.

FIG. 5B is a further enlarged fragmentary isometric view of the area within the square in FIG. 5A.

FIGS. 6A and 6B are views similar to FIGS. 5A and 5B, but showing the opposite side of the distal end of the probe.

FIG. 7 is a further enlargement of the distal end of the probe as shown in FIG. 5A, but with the tip omitted.

FIG. 8 is a further enlargement of the distal end of the probe as shown in FIG. 5B, but with the tip omitted.

FIG. 9 is an enlarged side view in elevation of the head of the probe and through which all connections are made.

FIG. 10 is an enlarged side view in elevation of the head of the probe, taken at 90° to FIG. 11.

FIG. 11 is a longitudinal sectional view taken along line 11-11 in FIG. 10.

FIG. 12 is a side view in elevation of the tip of the invention, shown removed from the probe shaft.

FIG. 13 is a longitudinal sectional view of the tip of FIG. 12.

FIG. 14 is an enlarged end view looking toward the proximal end of the probe shaft.

FIG. 15 is an enlarged end view looking toward the distal end of the probe shaft.

FIG. 16 is a side view in elevation of the sheath that is applied to the probe shaft in assembling the probe of the invention.

FIG. 17 is a longitudinal sectional view of the sheath of FIG. 16.

FIG. 18 is a side view in elevation of the probe shaft of the invention, shown with the sheath removed.

FIG. 19 is a longitudinal sectional view of the shaft of FIG. 18.

FIG. 20 is an enlarged fragmentary view looking at a slight angle toward the distal end of the probe shaft.

FIG. 21 is an enlarged fragmentary view similar to FIG. 20, but looking toward the opposite side of the shaft.

FIG. 22 shows a jamshidi needle being used for form a hole in a pedicle for receipt of a k-wire.

FIG. 23 shows a k-wire inserted and the jamshidi removed.

FIG. 24 shows the probe of the invention being placed, after which the k-wire is removed.

FIG. 25 shows the probe of the invention being manipulated to form a hole in the pedicle for receipt of a pedicle screw.

FIG. 26 shows the k-wire replaced and the probe being removed.

### DETAILED DESCRIPTION OF THE PREFERRED EMBODIMENTS

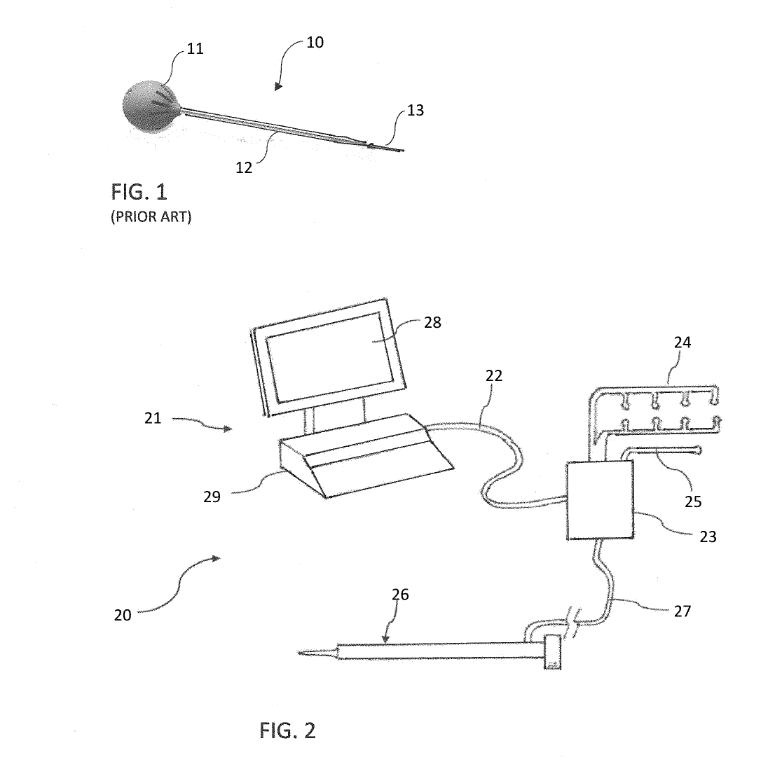

An awl as commonly used in the prior art to form a hole in a pedicle is indicated generally at 10 in FIG. 1. The awl has an enlarged head 11 at the proximal end for engagement with the hand of the surgeon, and an elongate shaft 12 terminating in a tip end 13 for forming the hole.

In accordance with the invention, either electromyography (EMG) or mechanomyography (MMG) may be used with the probe of the invention to alert the surgeon when a nerve is approached or a breach is about to occur. An MMG system generally is regarded as having a faster response and a higher sensitivity for detection of nerves at a lower threshold than does EMG. A suitable MMG system usable with the probe of the invention can be the Sentio MMG system available from Sentio LLC of Wixom, Mich.

A system as it might be constituted according to the invention when using either a mechanomyographic (MMG) monitoring system or an electromyographic monitoring (EMG) monitoring system is represented schematically at 20 in FIG. 2. The system would include a control unit 21 connected via a data cable 22 with a patient module 23. An EMG or MMG harness 24 and return electrode 25 are connected with the patient module, and a pedicle probe 26 according to the preferred form of the invention is also connected to the patient module via an electrical lead 27. The invention capitalizes on the insulating characteristics of bone, specifically that of the medial wall of the pedicle, and the conductivity of the adjacent nerve roots. That is, if the medial wall of the pedicle is breached or in danger of being breached, i.e., the layer of bone is too thin to provide enough insulation to prevent stimulation of adjacent nerves, a stimulation signal applied to the target site will cause the various muscle groups coupled to the nerve roots to react. The employment of electromyographic or mechanomyographic monitoring in the present invention to assess whether the muscle groups in the leg are innervating in response to the application of a stimulation signal does not require visual observation of twitching of the nerves.

In the case of an EMG system, the harness 24 relies on needles to detect subtle changes in electrical signals in muscle. In contrast, a mechanomyographic system such as the Sentio MMG® system employs proprietary accelerometer technology in the harness 24. These non-invasive accelerometer-based sensors measure MMG (mechanomyography) activity, or the mechanical “twitch” associated with muscle contraction.

With either MMG or EMG the control unit 21 includes a touch screen display 28 and a base 29, which collectively contain the essential processing capabilities for controlling the system 20. The data cable 22 establishes digital and/or analog electrical connections and communications between the control unit 21 and patient module 23. The main functions of the control unit 21 include receiving user commands via the touch screen display 28, activating stimulation, processing signal data according to defined algorithms as known in U.S. Pat. No. 8,255,044, for example, displaying received parameters and processed data, and monitoring system status and reporting fault conditions. The touch screen display 28 is preferably equipped with a graphical user interface (GUI) capable of communicating information to the user and receiving instructions from the user. The display 28 and/or base 29 may contain patient module interface circuitry that commands the stimulation sources, receives digitized signals and other information from the patient module 23, processes the EMG or MMG responses to extract characteristic information for each muscle group, and displays the processed data to the operator via the display 28.

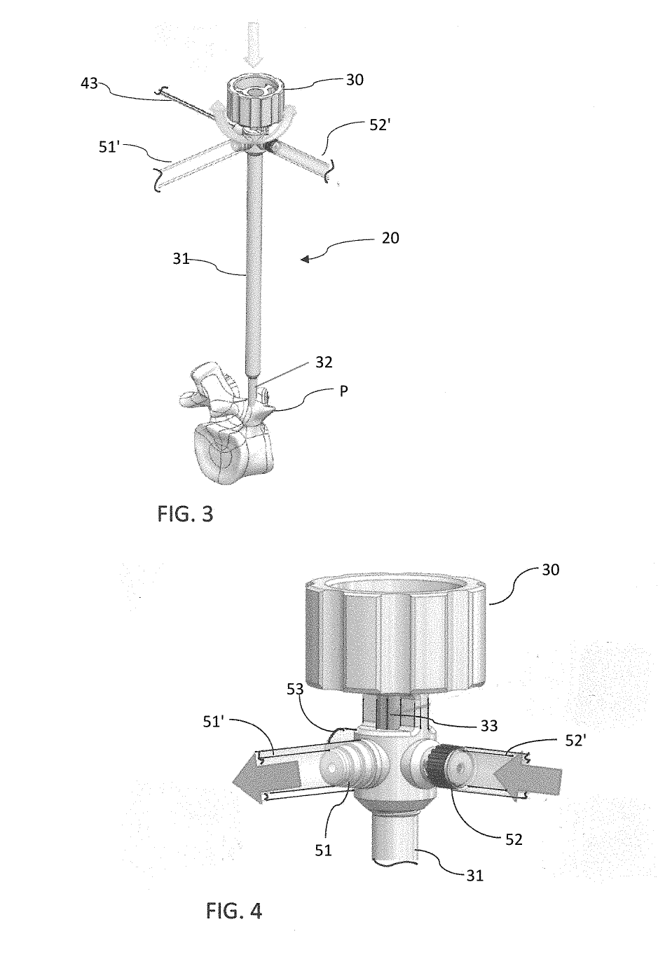

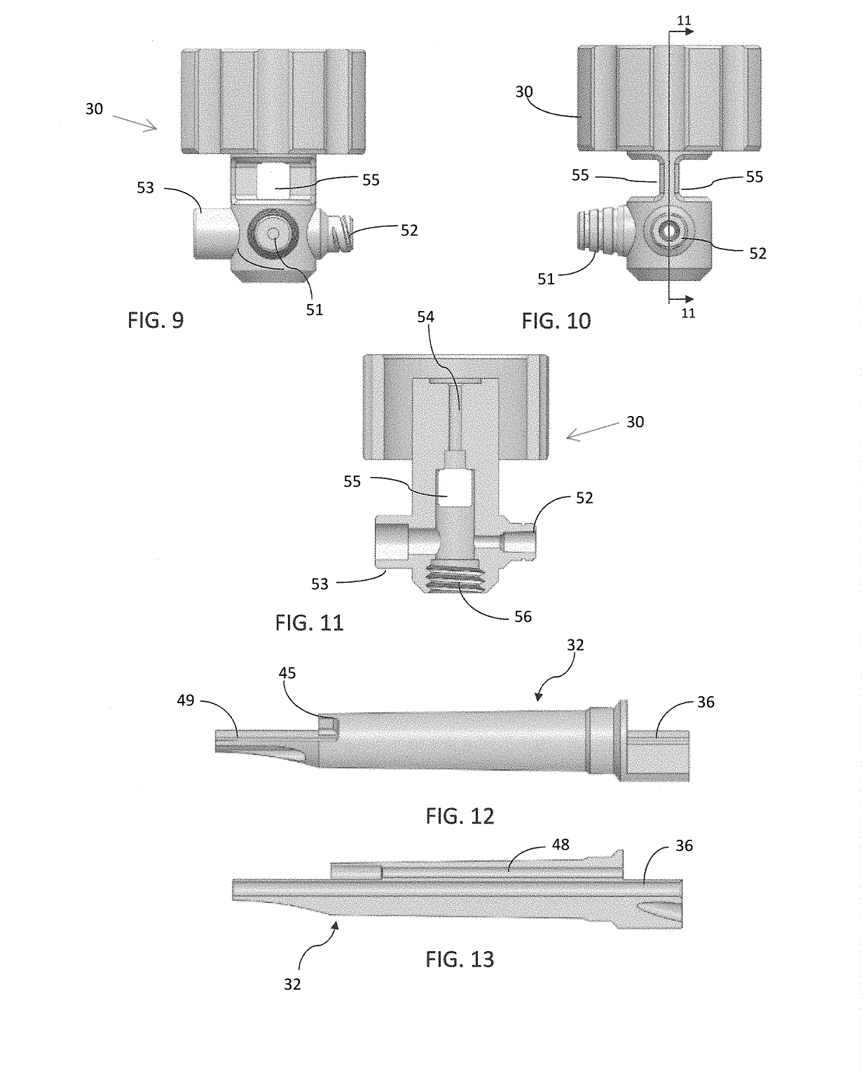

As seen in FIGS. 3-21, the probe 20 comprises a head 30 adapted to be held in the hand of the surgeon for manipulation of the probe, an elongate body 31 secured at its proximal end to the head, and a tip 32 secured to the distal end of the body for forming a hole in a pedicle P.

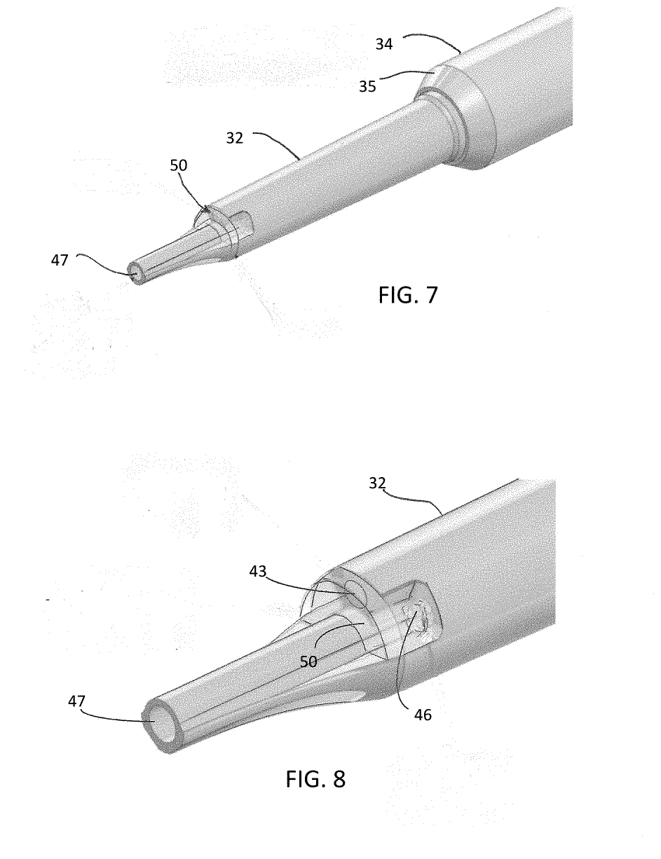

The body 31 is made up of a central shaft 33 enclosed in a sleeve or sheath 34. In a preferred embodiment the shaft and tip are made of an electrically conductive material (e.g. a suitable metal), and the sheath is made of an electrically non-conductive material (e.g. plastic). As shown in FIGS. 7 and 16-19, the sheath is slid over the shaft from the distal end of the shaft and the proximal end of the sheath is threaded at 34′ or otherwise configured for attachment to the head. An inturned lip or shoulder 35 at the distal end of the sleeve engages against the distal end of the shaft to hold the shaft rearwardly against the head.

The tip 32 is attached to the distal end of the shaft by engagement of a shaped end 36 on the tip with a complementally shaped opening and retainer 37 in the distal end of the shaft 33 (see FIGS. 12, 13 and 20, 21).

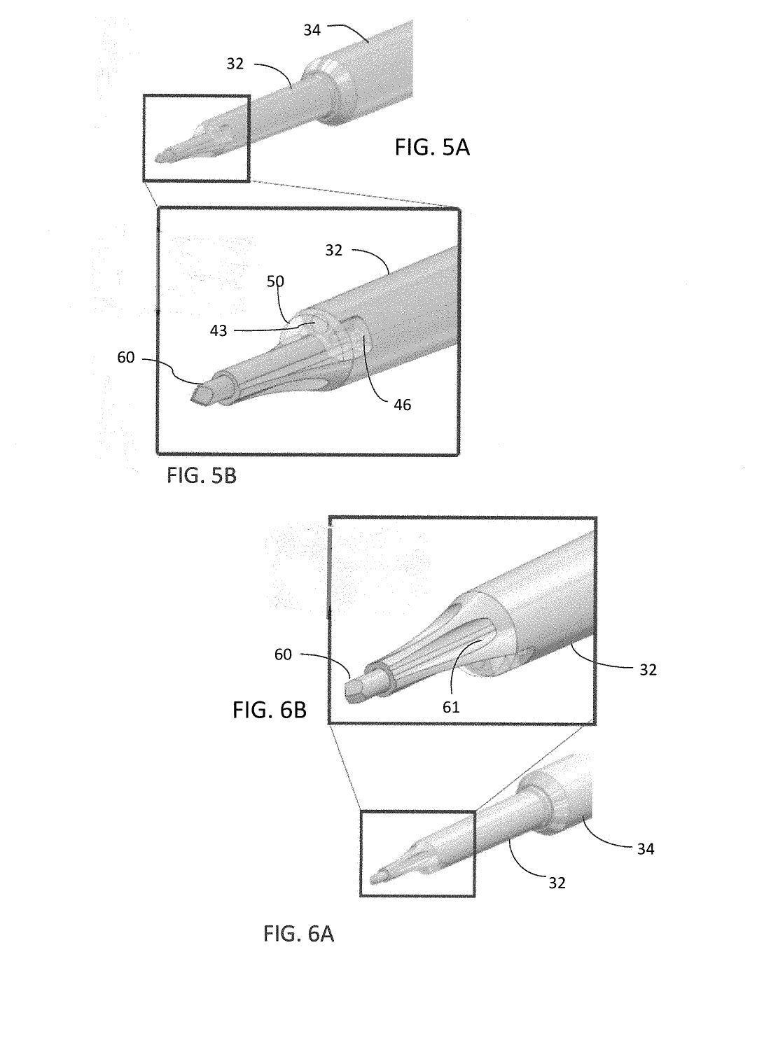

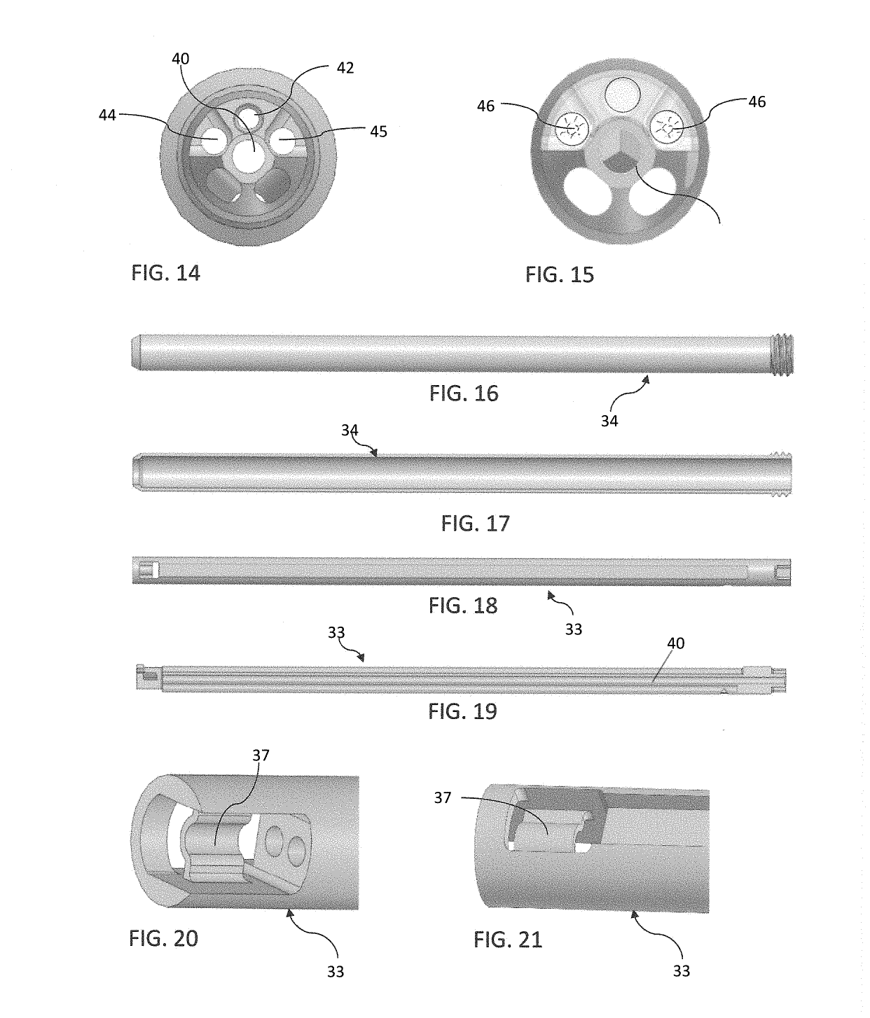

As seen best in FIGS. 14 and 19, a center bore 40 extends through the length of the shaft 33 for receiving a k-wire 41 (see FIGS. 23 and 24) during minimally invasive surgery (MIS). This bore also serves for supply of an irrigating fluid to the surgical site when the k-wire is removed, and for aspirating the irrigating fluid and debris from the site. A second bore 42 extends through the shaft at one side of the center bore for receiving an endoscopic camera 43 and third and fourth bores 44 and 45 are on opposite sides of the center bore for receiving fiber optic light bundles 46 to illuminate the surgical site (see FIGS. 5B, 6B, 8 and 14).

The tip 32 has corresponding bores aligned with the bores in the shaft. See, for example, the irrigation/aspiration bore 47 in FIG. 8 and bore 48 in FIG. 13 for receiving the camera. The distal end of the tip is recessed or cut away at 49 to provide clearance for the camera and lights, and a transparent shield 50 is positioned on the tip at the proximal end of the cut away area in overlying relationship to the camera and light source.

The head 30 has a plurality of lateral ports in its side, including a port 51 for connection with a source 51′ of irrigating fluid, a port 52 for connection to a suction source 52′ for aspiration of fluid and tissue away from the surgical site, and a port 53 for insertion of the endoscope 43 into the bore extending through the shaft. A longitudinal bore 54 extends through the center of the head for receiving a k-wire (see FIG. 11). Cut-outs 55 are provided in the area immediately above the lateral ports to expose the proximal end of the shaft 33 so that a variety of EMG/MMG clips can be attached to the shaft. In the particular example shown, the sleeve 31 is connected to the head by a threaded connection 56 in the bottom of the head (see FIG. 11). Irrigation ports 61 in the tip 32 (see FIG. 6B) double as cutting flutes.

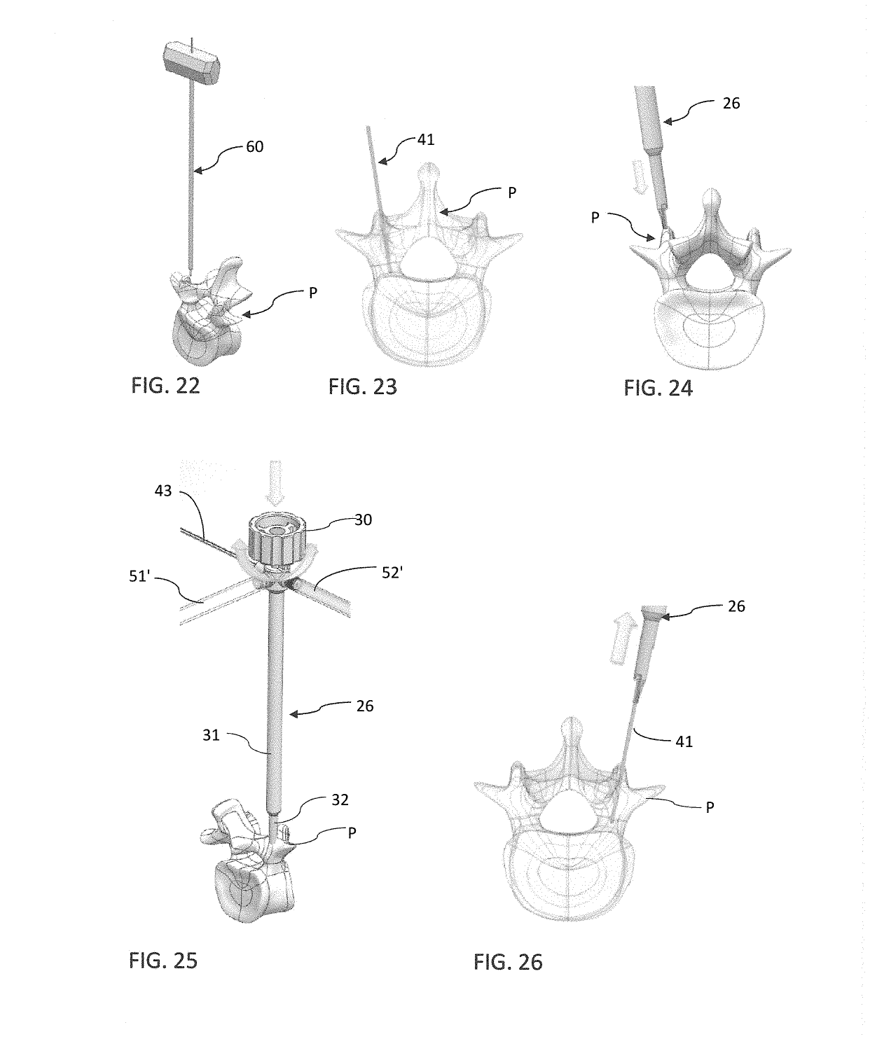

During a minimally invasive surgical procedure, depicted in FIGS. 22-26, a jamshidi 60 is used to form a pilot hole in a pedicle P (see FIG. 22), and a k-wire 41 is then inserted and the jamshidi removed (see FIG. 23). The probe of the invention is then put in place and the k-wire removed, as shown in FIG. 24. The surgeon then initiates irrigation and aspiration and applies axial pressure to the probe 26 while rotating it, visually observing until a safe and sufficient cannulation is present, as depicted in FIG. 25. The k-wire is then replaced and the probe removed as depicted in FIG. 26, after which the hole is tapped and a screw placed.

The endoscopic pedicle probe of the invention provides the surgeon with an illuminated, direct visual indication of the exact location of the probe and alerts the surgeon if a breach has occurred or is about to occur. It provides for flushing body fluids and debris away from the area being treated, whereby the hole can be formed with accuracy and precision.

The pedicle probe disclosed herein may be reusable, or the entire probe, inclusive or not inclusive of the endoscope, may be made disposable following a single use. Materials suitable for this purpose, such as hard plastics or carbon fiber, for example, may be used in the construction of the probe. In a preferred embodiment, as described herein, the probe shaft and tip are made of an electrically conductive material such as metal, and the sheath is made of a non-conductive material such as plastic.

While particular embodiments of the invention have been illustrated and described in detail herein, it should be understood that various changes and modifications may be made to the invention without departing from the spirit and intent of the invention as defined by the scope of the appended claims.

What is claimed is:

1. An endoscopic pedicle probe for use during spinal surgery to form a hole in a pedicle for reception of a pedicle screw, said probe comprising:
an elongate body having a proximal end and a distal end, a tip on the distal end that may be pushed through the pedicle to form the hole, and an enlarged head on the proximal end for cooperation with the hand of a surgeon to manipulate the probe;
said body comprising an inner shaft extending from the head to the tip;
an endoscope extending through an endoscope bore extending longitudinally through said shaft, said endoscope including a camera at said tip end, said camera being connected with a monitor to enable a surgeon to visually observe the area being treated;
light means extending through an illumination bore to illuminate the area being treated;
irrigation and aspiration means connected with an irrigation and aspiration bore to conduct an irrigating fluid to the area being treated and to aspirate the irrigating fluid and any debris away from the area being treated;
a cylindrical sleeve telescopically engaged over said shaft from the distal end to the proximal end; and
said sleeve being made of electrically non-conductive material and said shaft and tip being made of electrically conductive material.

2. The endoscopic pedicle probe as claimed in claim 1, wherein:
a plurality of lateral ports is in said head;
said endoscope extends through one of said ports to connect said camera to said monitor;
another of said ports connects with said illumination bore to connect a source of light with said light means; and
a further of said ports connects said irrigation and aspiration bore to a source of irrigating fluid and to a suction source for aspiration of fluid and tissue away from the area being treated.

3. The endoscopic pedicle probe as claimed in claim 2, wherein:
a longitudinal bore extends through the shaft and the head for receiving a k-wire.

4. The endoscopic pedicle probe as claimed in claim 3, wherein:
cut-outs in the area immediately above the lateral ports expose the proximal end of the shaft so that a variety of EMG/MMG clips can be attached to the shaft.

5. The endoscopic pedicle probe as claimed in claim 4, wherein:
the sleeve is connected to the head by a threaded connection in the bottom of the head, and the shaft is held to the head by the sleeve.

6. The endoscopic pedicle probe as claimed in claim 5, wherein:
said tip is attached to the distal end of the shaft by engagement of a shaped end on the tip with a complementally shaped opening and retainer in the distal end of the shaft.

7. The endoscopic pedicle probe as claimed in claim 6, wherein:
the tip has corresponding bores aligned with the bores in the shaft.

8. The endoscopic pedicle probe as claimed in claim 7, wherein:
the distal end of the tip is recessed to provide clearance for the camera and light means; and
a transparent shield is positioned on the tip at the proximal end of the recessed area in overlying relationship to the camera and light source.

9. The endoscope and pedicle probe as claimed in claim 8, wherein:
irrigation ports in the tip double as cutting flutes.

10. The endoscope and pedicle probe as claimed in claim 9, wherein:
the sheath is slid over the shaft from the distal end of the shaft and the proximal end of the sheath is threaded for attachment to the head; and
an inturned lip at the distal end of the sleeve engages against the distal end of the shaft to hold the shaft rearwardly against the head.

11. An endoscopic pedicle probe for use during spinal surgery to form a hole in a pedicle for reception of a pedicle screw, comprising:
an elongate body having a proximal end and a distal end;
a tip at the distal end that may be pushed into the pedicle to form the hole; and
an enlarged head on the proximal end for cooperation with the hand of a surgeon to manipulate the probe;
an endoscope and light extending through the body, said endoscope being connected with a monitor to enable a surgeon to visually observe the area being treated; and
a mechanomyographic monitoring system connected with the probe to perform real time dynamic pedicle integrity assessments during a procedure to detect a breach or potential breach of the pedicle and alert the surgeon.

12. The endoscopic pedicle probe as claimed in claim 11, wherein:
said body comprises an inner shaft extending from the head to the tip, and a cylindrical sleeve telescopically engaged over said shaft from the distal end to the proximal end.

13. The endoscopic pedicle probe as claimed in claim 12, wherein:
said shaft and tip are made of an electrically conductive material and said sleeve is made of an electrically non-conductive material.

14. The endoscopic pedicle probe as claimed in claim 13, wherein:
said shaft has a plurality of longitudinal bores extending therethrough from the proximal end to the distal end; and
means is connected with one of said bores to conduct an irrigating fluid to the area being treated and to aspirate the irrigating fluid and any debris from the area.

15. The endoscopic pedicle probe as claimed in claim 14, wherein:
said endoscope and light extend through another of said bores.

16. The endoscopic pedicle probe as claimed in claim 15, wherein:
a laterally extending port in said head connects with said another of said bores; and
said endoscope and light extend through said laterally extending port to said monitor.

17. The endoscopic pedicle probe as claimed in claim 16, wherein:
another laterally extending port in said head connects a source of irrigating fluid with said one bore to conduct an irrigating fluid to said one bore.

18. The endoscopic pedicle probe as claimed in claim 17, wherein:
a source of suction is also connected with said another laterally extending port to aspirate irrigating fluid and debris through said one bore and away from the area being treated.

19. The endoscopic pedicle probe as claimed in claim 18, wherein:
cut-outs are in said head in position to expose the proximal end of said shaft and enable electrical conductors to be connected to said shaft.
